(19) United States
(12) Patent Application Publication
(10) Pub. No.: US 2006/0217535 A1
(43) Pub. Date: Sep. 28, 2006

(54) Hemipteran muscarinic receptor

(76) Inventors: Debra Yuhas, Wrightstown, NJ (US); Cancan Huang, Kendall Park, NJ (US); Susan Gilbey, Camden, ME (US); Valerie Deutsch, Mercerville, NJ (US); Jeffrey D. Herron, Bordentown, NJ (US); Ruihua Chen, Kendall Park, NJ (US)

Correspondence Address: PATENT ADMINISTRATOR;FMC CORPORATION, 1735 MARKET STREET, PHILADELPHIA, PA, 19103, US

(21) Appl. No.: 10/512,375

(22) PCT Filed: May 9, 2003
(86) PCT No.: PCT/US03/14593

§ 371 Date: Jun. 30, 2005

Related U.S. Application Data
(60) Provisional application No. 60/379,220, filed on May 9, 2002.
(60) Provisional application No. 60/401,608, filed on Aug. 7, 2002.

(51) Int. Cl. C07K 14/435; C07H 21/04; C12P 21/06; C12N 5/06

(52) U.S. Cl. 530/350; 530/387.1; 435/69.1; 435/320.1; 435/348; 536/23.5

(57) ABSTRACT

Novel nucleic acid sequences encoding hemipteran muscarinic receptors, and recombinant expressions and host cells comprising the same are disclosed. Isolated hemipteran muscarinic receptors, host cells expressing hemipteran muscarinic receptors, methods of producing the hemipteran muscarinic receptors and antibodies specific for hemipteran muscarinic receptors are also disclosed. Methods of identifying modulators of hemipterana muscarinic receptors are disclosed.

This application claims the benefit of U.S. Provisional Application No. 60/379,220, filed May 9, 2002, and U.S. Provisional Application No. 60/401,608, filed Aug. 7, 2002.

### FIELD OF THE INVENTION

The present invention relates to nucleotide sequences that are useful in agrochemical, veterinary or pharmaceutical fields. In particular, the invention relates to nucleotide sequences that encode or may be used to express amino acid sequences that are useful in the identification or development of compounds with (potential) activity as pesticides or as pharmaceuticals. Even more particularly, the invention also relates to the amino acid sequences—such as proteins or polypeptides—that are encoded by, or that may be obtained by suitable expression of, the nucleotide sequences of the invention.

### BACKGROUND OF THE INVENTION

Acetylcholine (“Ach”) is a major excitory neurotransmitter in the insect CNS and periphery; mediating signal transmission through cholinergic receptors, which are, comprised of two main classes nicotinic and muscarinic receptors (Florey et al., Can. J. Biochem. Physiol., 41, 2619-2626, 1983; and Eldefrawi et al., Receptors for Neurotransmitters, Hormones and Pheromones in Insects. 5). Muscarinic receptors (i.e., mAchR receptor) have been studied and postulated as a potential site of action for new insecticides since the 1980s when muscarinic antagonist ligands such as quinuclidinyl benzilate (“QNB”) were shown to bind to fractionated nervous tissue from insects (Schmidt Nelsen, J. Neurochem. 20, 1013-1029, 1977; Eldefawi et al., Putative acetylcholine receptors, in housefly brain. Receptors for Neurotransmitters, Hormones and Pheromones in Insects ed Satel, Hall and Hodebrand, 59-70 Elsevier; Dudai et al., FEBS Lett., 81, 134-6, 1977). Muscaranic agonists such as oxotremorine and antagonists such as QNB were also found to affect AchR release in locust synaptomes. (Breer Comp. Biochem. Physiol., 90C, No. 1, pp. 275-280, 1988). The muscarinic receptors were also of interest given the significance of the nicotinic receptors as a target for insecticides. The nicotinic receptors are ligand gated ion channels and are the site of action for several insecticides such as imidacloprid. In the 1990's electrophysiological measurement and characterization of the activity of mAchR agonists and antagonists in insect preparations (Trimmer et al., Trends in Neuroscience, 18, No. 2, p 104-10, 1995) and insect activity of muscarinic agonists on mites and aphids was reported (Dick et al., Pesticide Science, 49, 268-76, 1997).

In the 1989 the Drosophila muscarinic receptor was cloned and found to be functional when expressed in oocytes and in mammalian cells (Onai et al., FEBS Lett., 255, 219-225 (1989); Shapiro et al., Proc. Natl. Acad. Sci., 86 (22), 9039-9043 (1989)). Isolation and expression of the Drosophila muscarinic receptor has established its function as a muscarinic receptor, though it has less than 35% homology to any of the subunits that constitute the functional analogue in vertebrates. Partial sequences from the tobacco hornworm have also been isolated and cloned (Wang and Trimmer, dissertation, Muscarinic Acetylcholine Receptor Heterogeneity in the Central Nervous System of Tobacco Hornworm, 1998, UMI Company, Ann Arbor Mich. UMI#991539).

### SUMMARY OF THE INVENTION

One embodiment of the invention relates to nucleotide sequences that encode or may be used to express amino acid sequences that are useful in the identification or development of compounds with (potential) activity as pesticides or as pharmaceuticals. These nucleotide sequences, including mutants and fragments thereof, which will be further described below, will also be referred to herein as “nucleotide sequences of the invention”.

Another embodiment of the invention relates to the amino acid sequences—such as proteins or polypeptides—that are encoded by, or that may be obtained by suitable expression of, the nucleotide sequences of the invention. These amino acid sequences, including mutants and fragments thereof, which will be further described below, will also be referred to herein as “amino acid sequences of the invention”.

Yet another embodiment of the invention relates to the use of the nucleotide sequences of the invention, preferably in the form of a suitable genetic construct as described below, in the transformation of host cells or host organisms, for example for the expression of the amino acid sequences of the invention. The invention also relates to host cells or host organisms that have been transformed with the nucleotide sequences of the invention or that can express the amino acid sequences of the invention.

In still yet another embodiment, the invention relates to methods for the identification or development of compounds that can modulate the biological activity of the amino acid sequences of the invention, in which the above mentioned nucleotide sequences, amino acid sequences, genetic constructs, host cells or host organisms are used. Such methods, which will usually be in the form of an assay or screen, will also be further described below.

### Definitions

Collectively, the nucleic acids of the present invention will be referred to herein as “nucleic acids of the invention”. Also, where appropriate in the context of the further description of the invention below, the terms “nucleotide sequence of the invention” and “nucleic acid of the invention” may be considered essentially equivalent and essentially interchangeable.

Also, for the purposes of the present invention, a nucleic acid or amino acid sequence is considered to “(in) essentially isolated (form)”—for example, from its native biological source—when it has been separated from at least one other component (and in particular macromolecule) with which it is usually associated, such as another nucleic acid, another protein or polypeptide or another (polymeric) biological component. In particular, a nucleic acid or amino acid sequence is considered “essentially isolated” when it has been purified at least 2-fold, in particular at least 10-fold, more in particular at least 100-fold, and up to 1000-fold or more.

### BRIEF DESCRIPTION OF THE DRAWINGS

FIG. 1A shows the suspected protein that encodes the mAchR receptor of Aphis gossypii.

### DETAILED DESCRIPTION OF THE INVENTION

The present invention was established from the finding that the amino acid sequences of the invention can be used as (potential) “target(s)” for in vitro or in vivo interaction with chemical compounds and other factors (with the term “target” having its usual meaning in the art, provide for example the definition given in WO 98/06737). Consequently, compounds or factors that have been identified as interacting with the amino acid sequences of the invention (e.g. by the methods as described herein below) may be useful as active agents in the agrochemical, veterinary or pharmaceutical fields, see Example 4 set forth below.

In one embodiment, the invention relates to a nucleic acid, preferably in (essentially) isolated form, which nucleic acid comprises a nucleotide sequence of the invention, and in particular the nucleotide sequences of SEQ ID NO: 1 and SEQ ID NO: 3. The nucleotide sequence of SEQ ID NO: 1 or SEQ ID NO: 3, preferably SEQ ID NO: 1, was derived or isolated from the Aphis gossypii organism, in the manner as further described in the Experimental Part below.

Generally, the nucleotide sequences of the invention, when in the form of a nucleic acid, may be DNA or RNA, and may be single stranded or double stranded. For example, the nucleotide sequences of the invention may be genomic DNA, cDNA or synthetic DNA (such as DNA with a codon usage that has been specifically adapted for expression in the intended host cell or host organism, which may for instance be designed using suitable computer programs such as Oligo-4, available from National Biosciences, Inc. (Plymouth, Minn.) and Consensus-Degenerate Hybrid Oligonucleotide Primers Software (CODEHOP) from Henikoff et al. (Nucleic Acids Research, 26, 70, 1628-1635, 1998) available on line through the Fred Hutchinson Cancer Research Center. Thus, the nucleotide sequences of the invention may contain intron sequences, and also generally comprises different splice variants.

Yet another embodiment relates to a double stranded RNA molecule directed against a nucleotide sequence of the invention (one strand of which will usually comprise at least part of a nucleotide sequence of the invention). The invention also relates to genetic constructs that can be used to provide such double stranded RNA molecules (e.g. by suitable expression in a host cell or host organism, or for example in a bacterial strain such as E. coli). For such constructs, reference is made to Maniatis et al., Molecular Cloning, a Laboratory Manual (Cold Spring Harbor Press, 1989).

In a broader sense, the term “nucleotide sequence of the invention” also comprises:
parts or fragments of the nucleotide sequence of SEQ ID NO: 1 or SEQ ID NO: 3;
(natural or synthetic) mutants, variants, alleles, analogs, orthologs (herein below collectively referred to as “mutants”) of the nucleotide sequence of SEQ ID NO: 1 or SEQ ID NO: 3, as further described below.
parts or fragments of such (natural or synthetic) mutants;
nucleotide fusions of the nucleotide sequence of SEQ ID NO: 1 or SEQ ID NO: 3 (or a part or fragment thereof) with at least one further nucleotide sequence;
nucleotide fusions of (natural or synthetic) mutants (or a part or fragment thereof) with at least one further nucleotide sequence;
   in which such mutants, parts, fragments or fusions are preferably as further described below.

The invention also comprises different splice variants of the above nucleotide sequences.

Preferably, a nucleotide sequence of the invention will have a length of at least 500 nucleotides, preferably at least 1,000 nucleotides, more preferably at least 1,500 nucleotides; and up to a length of at most 2,000 nucleotides, preferably at most 1,800 nucleotides, more preferably at most 1,700 nucleotides.

Examples of parts or fragments of the nucleotide sequence of SEQ ID NO: 1 or SEQ ID NO: 3, preferably SEQ ID NO: 1; or a part or fragment of a (natural or synthetic) mutant thereof include, but are not limited to, 5′ or 3′ truncated nucleotide sequences, or sequences with an introduced in frame start codon or stop codon. Also, two or more such parts or fragments of one or more nucleotide sequences of the invention may be suitably combined (e.g. ligated in frame) to provide a further nucleotide sequence of the invention.

Preferably, any such parts or fragments will be such that they comprise at least one continuous stretch of at least 100 nucleotides, preferably at least 250 nucleotides, more preferably at least 500 nucleotides, even more preferably more than 100 nucleotides, of the nucleotide sequence of SEQ ID NO: 1 or SEQ ID NO: 3, preferably SEQ ID NO: 1.

Also, it is expected that—based upon the disclosure herein—the skilled person will be able to identify, derive or isolate natural “mutants” (as mentioned above) of the nucleotide sequence of SEQ ID NO: 1 or SEQ ID NO: 3, preferably SEQ ID NO: 1 from (other individuals of) the same species (for example from an individual of a different strain or line, including but not limited to mutant strains or lines). It is also expected that—based upon the disclosure herein—the skilled person will be able to provide or derive synthetic mutants (as defined hereinabove) of the nucleotide sequence of SEQ ID NO: 1 or SEQ ID NO: 3, preferably SEQ ID NO: 1.

In one specific embodiment, the mutant is such that it encodes the nucleotide sequence of SEQ ID NO: 1 or a part or fragment thereof.

Preferably, any mutants as described herein will have one or more, and preferably all, of the structural characteristics or conserved features referred to below for the nucleotide sequences of SEQ ID NO: 1 and SEQ ID NO: 3.

In particular, any mutants, parts or fragments as described herein may be such that they at least encode the active or catalytic site of the corresponding amino acid sequence of the invention and a binding domain of the corresponding amino acid sequence of the invention.

Also, any mutants, parts or fragments as described herein will preferably have a degree of “sequence identity”, at the nucleotide level, with the nucleotide sequence of SEQ ID NO 1 or SEQ ID NO: 3, preferably SEQ ID NO: 1, of at least 75%, preferably at least 80%, more preferably at least 85%, and in particular more than 90%, and up to 95% or more.

Also, preferably, any mutants, parts or fragments of the nucleotide sequence of the invention will be such that they encode an amino acid sequence which has a degree of “sequence identity”, at the amino acid level, with the amino acid sequence of SEQ ID NO: 2 or SEQ ID NO: 4, preferably SEQ ID NO: 2, of at least 50%, preferably at least 60%, more preferably at least 70%, even more preferably at least 80%, and in particular more than 90% and up to 95% or more, in which the percentage of “sequence identity” is calculated as described below.

For this purpose, the percentage of “sequence identity” between a given nucleotide sequence and the nucleotide sequence of SEQ ID NO: 1 or SEQ ID NO: 3 may be calculated by dividing the number of nucleotides in the given nucleotide sequence that are identical to the nucleotide at the corresponding position in the nucleotide sequence of SEQ ID NO: 1 or SEQ ID NO: 3 by the total number of nucleotides in the given nucleotide sequence and multiplying by 100%, in which each deletion, insertion, substitution or addition of a nucleotide—compared to the sequence of SEQ ID NO: 1 or SEQ ID NO:3—is considered as a difference at a single nucleotide position.

Alternatively, computer programs for determining sequence identity are publicly available. A preferred computer program for determining sequence identity is the program in Geneworks v 2.5 (Intelligenetics Inc, Mountain View Calif.), which uses a progressive alignment procedure similar to FASTA. Preferably the parameters used with the Geneworks program are: cost to open gap=50, lengthen gap=100, minimum diagonal length=4, maximum diagonal offset=125. Other computer program methods to determine identity between two sequences include, but are not limited to, GCG program package (Devereux, J., et al., Nucleic Acids Research 12(1): 387 (1984)), BLASTP, BLASTN, FASTA (Atschul, S. F. et al., J. Molec. Biol. 215: 403-410 (1990)) and Vector NTI (InforMax Inc., Bethesda, Md.). The BLAST X program is publicly available from NCBI (blast@ncbi.nlm.nih.gov) and other sources (BLAST Manual, Altschul et al., NCBI NLM NIH, Bethesda, Md. 20894; Altschul et al., J. Mol. Biol. 215: 403-410 (1990)), Vector NTI Suite Version 6 available from Informax Inc. North Bethesda, Md.

Also, in a preferred aspect, any mutants, parts or fragments as described herein will encode proteins or polypeptides having biological activity that is essentially similar to the biological activity described above for the sequences of SEQ ID NO: 1 or SEQ ID NO: 3, i.e. to a degree of at least 50%, preferably at least 75%, and up to 90%, as measured by the assay mentioned above.

Any mutants, parts or fragments as described herein are preferably such that they are capable of hybridizing with the nucleotide sequence of SEQ ID NO: 1 or SEQ ID NO: 3, preferably SEQ ID NO: 1, i.e. under conditions of “moderate stringency”, and preferably under conditions of “high stringency”. Such conditions will be clear to the skilled person, for example from the standard handbooks, such as Sambrook et al. and Ausubel et al., mentioned above, as well as in EP 0 967 284, EP 1 085 089 or WO 00/55318.

It is also within the scope of the invention to use a fusion of a nucleotide sequence of the invention (as described above) with one or more further nucleotide sequence(s), including but not limited to one or more coding sequences, non-coding sequences or regulatory sequences. Preferably, in such fusions, the one or more further nucleotide sequences are operably connected (as described below) to the nucleotide sequence of the invention (for example so that, when the further nucleotide sequence is a coding sequence, the nucleotide fusion encodes a protein fusion as described below).

In another embodiment, the invention relates to an antisense molecule against a nucleotide sequence of the invention.

The nucleic acids of the invention may also be in the form of a genetic construct, again as further described below. Genetic constructs of the invention will generally comprise at least one nucleotide sequence of the invention, optionally linked to one or more elements of genetic constructs known per se, as described below. Such genetic constructs may be DNA or RNA, and are preferably double-stranded DNA. The constructs may also be in a form suitable for transformation of the intended host cell or host organism, in a form suitable for integration into the genomic DNA of the intended host cell or in a form suitable independent replication, maintenance and inheritance in the intended host organism. For instance, the genetic construct may be in the form of a vector, such as for example a plasmid, cosmid, a yeast artificial chromosome (“YAC”), a viral vector or transposon. In particular, the vector may be an expression vector, i.e. a vector that can provide for expression in vitro or in vivo (e.g. in a suitable host cell or host organism as described below). An expression vector comprising a nucleotide sequence of the invention is also referred to herein as a recombinant expression vector. These constructs will also be referred to herein as “genetic constructs of the invention”.

In a preferred embodiment, such a construct a recombinant expression vector which will comprise:
a) the nucleotide sequence of the invention; operably connected to:
b) one or more regulatory elements, such as a promoter and optionally a suitable terminator;
   and optionally also:
c) one or more further elements of genetic constructs known per se; in which the terms “regulatory element”, “promoter”, “terminator”, “further elements” and “operably connected” have the meanings indicated herein below.

As the one or more “further elements” referred to above, the genetic construct(s) of the invention may generally contain one or more suitable regulatory elements (such as a suitable promoter(s), enhancer(s), or terminator(s)), 3′- or 5′-untranslated region(s) (“UTR”) sequences, leader sequences, selection markers, expression markers or reporter genes, or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and the transformation technique to be used.

Preferably, in the genetic constructs of the invention, the one or more further elements are “operably linked” to the nucleotide sequence(s) of the invention or to each other, by which is generally meant that they are in a functional relationship with each other. For instance, a promoter is considered “operably linked” to a coding sequence if said promoter is able to initiate or otherwise control or regulate the transcription or the expression of a coding sequence (in which said coding sequence should be understood as being “under the control of” said promoter)

Generally, when two nucleotide sequences are operably linked, they will be in the same orientation and usually also in the same reading frame. They will usually also be essentially contiguous, although this may also not be required.

Preferably, the optional further elements of the genetic construct(s) used in the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling or regulating the transcription or the expression of a nucleotide sequence—e.g. a coding sequence—to which it is operably linked (as defined above).

Such a promoter may be a constitutive promoter or an inducible promoter, and may also be such that it (only) provides for expression in a specific stage of development of the host cell or host organism, or such that it (only) provides for expression in a specific cell, tissue, organ or part of a multicellular host organism.

Some particularly preferred promoters include, but are not limited to, constitutive promoters, such as cytomegalovirus (“CMV”), Rous sarcoma virus (“RSV”), simian virus-40 (“SV40”), for example, pSVL SV40 Late Promoter Expression Vector (Pharmacia Biotech Inc., Piscataway, N.J.), or herpes simplex virus (“HSV”) for expression in mammalian cells or insect constituitive promoters such a the immediate early baculovirus promoter described by Jarvis et al. Methods in Molecular Biology Vol. 39 Baculovirus Expression Protocols ed. C. Richardson. Hamana Press Inc., Totowa, N.J. 1995 available in pIE vectors from Novagen (Novagen, Inc. Madison, Wis.) or insect inducible promoters such as the Drosophila metallothionein promoter described by Bunch et al. Nucleic Acids Research, Vol. 6 No. 3 1043-106, 1988 available in vectors from Invitrogen, (Invitrogen Corporation, Carlsbad, Calif.).

A selection marker should be such that it allows—i.e. under appropriate selection conditions—host cells or host organisms that have been (successfully) transformed with the nucleotide sequence of the invention to be distinguished from host cells or organisms that have not been (successfully) transformed. Some preferred, but non-limiting examples of such markers are genes that provide resistance against antibiotics (such as geneticin or G-418 (GIBCO-BRL, Grand Island, N.Y.), hygromycin, kanamycin or ampicillin), genes that provide for temperature resistance, or genes that allow the host cell or host organism to be maintained in the absence of certain factors, compounds or (food) components in the medium that are essential for survival of the non-transformed cells or organisms.

A leader sequence should be such that—in the intended host cell or host organism—it allows for the desired post-translational modifications or such that it directs the transcribed mRNA to a desired part or organelle of a cell such as a signal peptide. A leader sequence may also allow for secretion of the expression product from said cell. As such, the leader sequence may be any pro-, pre-, or prepro-sequence operable in the host cell or host organism, including, but not limited to, picornavirus leaders, potyvirus leaders, a human immunoglobulin heavy-chain binding protein (“BiP”), a tobacco mosaic virus leader (“TMV”), and a maize chlorotic mottle virus leader (“MCMV”).

An expression marker or reporter gene should be such that—in the host cell or host organism—it allows for detection of the expression of (a gene or nucleotide sequence present on) the genetic construct. An expression marker may optionally also allow for the localization of the expressed product, e.g. in a specific part or organelle of a cell or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins, such as GFP, antibody recognition proteins, for example, V5 epitope or poly Histidine available in vectors and antibodies supplied by Invitrogen (Invitrogen, Carlsbad, Calif.), or purification affinity handles such as polyhistidine which allows for purification on nickel columns or dihydrofolate reductase which allows for purification on methotrexate column, or markers which allow for selection of cells expressing the gene such as the E. coli beta-galactosidase gene.

For some non-limiting examples of the promoters, selection markers, leader sequences, expression markers and further elements that may be present or used in the genetic constructs of the invention—such as terminators, transcriptional or translational enhancers or integration factors—reference is made to the general handbooks such as Sambrook et al. and Ausubel et al. mentioned above, to W. B. Wood et al., “The nematode Caenorhabditis elegans”, Cold Spring Harbor Laboratory Press (1988) and D. L. Riddle et al., “C. ELEGANS II”, Cold Spring Harbor Laboratory Press (1997), as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089. Other examples will be clear to the skilled person.

Another embodiment of the invention relates to a host cell or host organism that has been transformed or contains with a nucleotide sequence, with a nucleic acid or with a genetic construct of the invention. The invention also relates to a host cell or host organism that expresses, or (at least) is capable of expressing (e.g. under suitable conditions), an amino acid sequence of the invention. Collectively, such host cells or host organisms will also be referred to herein as “host cells or host organisms of the invention”.

The host cell may be any suitable (fungal, prokaryotic or eukaryotic) cell or cell line, for example:
a bacterial strain, including but not limited to strains of E. coli, Bacillus, Streptomyces and Pseudomonas;
a fungal cell, including but not limited to cells from species of Aspergillus and Trichoderma;
a yeast cell, including but not limited to cells from species of Kluyveromyces or Saccharomyces;
an amphibian cell or cell line, such as Xenopus oocytes.

In one specific embodiment, which may particularly useful when the nucleotide sequences of the invention are (to be) used in the discovery and development of insecticidal compounds, the host cell may be an insect-derived cell or cell line, such as:
cells or cell lines derived from lepidoptera, including but not limited to Spodoptera frugiperda, such as, SF9 and Sf21 cells,
cells or cell lines derived from Aphis;
cells or cell lines derived from Drosophila, such as Schneider and Kc cells; and
cells or cell lines derived from a pest species of interest (as mentioned below), such as from Heliothis virescens.

The host cell may also be a mammalian cell or cell line, including but not limited to CHO- and BHK-cells and human cells or cell lines such as HeK, HeLa and COS.

The host organism may be any suitable multicellular (vertebrate or invertebrate) organism, including but not limited to:
a nematode, including but not limited to nematodes from the genus Caenorhabditis, such as C. elegans,
an insect, including but not limited to species of Aphis, Drosophila or a specific pest species of interest (such as those mentioned above);
other well known model organisms, such as zebrafish;
a mammal such as a rat or mouse;

Other suitable host cells or host organisms will be clear to the skilled person, for example from the handbooks and patent applications mentioned above.

It should be noted that when a nucleotide sequence of the invention is expressed in a multicellular organism, it may be expressed throughout the entire organism, or only in one or more specific cells, tissues, organs or parts thereof, for example by expression under the control of a promoter that is specific for said cell(s), tissue(s), organ(s) or part(s).

The nucleotide sequence may also be expressed during only a specific stage of development or life cycle of the host cell or host organism, again for example by expression under the control of a promoter that is specific for said stage of development or life cycle. Also, as already mentioned above, said expression may be constitutive, transient or inducible.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and offspring of the host cell or host organism of the invention, which may for instance be obtained by cell division or by sexual or asexual reproduction.

In yet another aspect, the invention relates to a nucleic acid, preferably in (essentially) isolated form, which nucleic acid encodes or can be used to express an amino acid sequence of the invention (as defined herein), and in particular the amino acid sequence of SEQ ID NO: 2 and SEQ ID NO: 4. A particularly preferred example of an amino acid sequence of the invention is the amino acid sequence of SEQ ID NO 2.

The amino acid sequence of SEQ ID NO: 2 or SEQ ID NO: 4, preferably SEQ ID NO: 2, may be isolated from the species mentioned above, using any technique(s) for protein isolation and purification known to one skilled in the art. Alternatively, the amino acid sequence of SEQ ID NO: 2 or SEQ ID NO: 4 may be obtained by suitable expression of a suitable nucleotide sequence—such as the nucleotide sequence of SEQ ID NO: 1 or SEQ ID NO: 3 or a suitable mutant thereof—in an appropriate host cell or host organism, as further described below.

In another aspect, the invention relates to a protein or polypeptide, preferably in (essentially) isolated form, said protein or polypeptide comprising an amino acid sequence of the invention (as defined above), in particular the amino acid sequence of SEQ ID NO: 2 or SEQ ID NO: 4, more particularly the amino acid sequence of SEQ ID NO: 2.

In a broader sense, the term “amino acid sequence of the invention” also comprises:
parts or fragments of the amino acid sequence of SEQ ID NO: 2 or SEQ ID NO: 4;
(natural or synthetic) mutants, variants, alleles, analogs, orthologs (herein below collectively referred to as “analogs”) of the amino acid sequence of SEQ ID NO: 2 or SEQ ID NO: 4;
parts or fragments of such analogs;
fusions of the amino acid sequence of SEQ ID NO:2 or SEQ ID NO:4 (or a part or fragment thereof) with at least one further amino acid residue or sequence;
fusions of the amino acid sequence of an analog (or a part or fragment thereof) with at least one further amino acid residue or sequence;
   in which such mutants, parts, fragments or fusions are preferably as further described below.

The term “amino acid sequence of the invention” also comprises “immature” forms of the abovementioned amino acid sequences, such as a pre-, pro- or prepro-forms or fusions with suitable leader sequences. Also, the amino acid sequences of the invention may have been subjected to post-translational processing or be suitably glycosylated, depending upon the host cell or host organism used to express or produce said amino acid sequence; or may be otherwise modified (e.g. by chemical techniques known per se in the art).

Examples of parts or fragments of the amino acid sequence of SEQ ID NO: 2 or SEQ ID NO: 4, or a part or fragment of a (natural or synthetic) analog thereof mutant thereof include, but are not limited to, N- and C-truncated amino acid sequence. Also, two or more parts or fragments of one or more amino acid sequences of the invention may be suitably combined to provide an amino acid sequence of the invention.

Preferably, an amino acid sequence of the invention has a length of at least 100 amino acids, preferably at least 250 amino acids, more preferably at least 500 amino acids; and up to a length of at most 800 amino acids, preferably at most 750 amino acids, more preferably at most 600 amino acids.

Preferably, any such parts or fragments will be such that they comprise at least one continuous stretch of at least 5 amino acids, preferably at least 10 amino acids, more preferably at least 20 amino acids, even more preferably more than 30 amino acids, of the amino acid sequence of SEQ ID NO: 2 or SEQ ID NO: 4.

In particular, any parts or fragments as described herein are such that they (at least) comprise the active or catalytic site of the corresponding amino acid sequence of the invention or a binding domain of the corresponding amino acid sequence of the invention. As will be clear to the skilled person, such parts or fragments may find particular use in assay- and screening techniques (as generally described below) and (when said part or fragment is provided in crystalline form) in X-ray crystallography.

Also, it is expected that—based upon the disclosure herein—the skilled person will be able to identify, derive or isolate natural “analogs” (as mentioned above) of the amino acid sequence of SEQ ID NO: 2 or SEQ ID NO: 4. Such mutants could be derived from (other individuals of) the same species (for example from an individual of a different strain or line, including but not limited to mutant strains or lines); or from (individuals of) other species. For example, such analogs could be derived from the insect species mentioned above.

It is also expected that—based upon the disclosure herein—the skilled person will be able to provide or derive synthetic “analogs” (as mentioned above) of the amino sequence of SEQ ID NO: 2 or SEQ ID NO: 4.

Preferably, any mutants as described herein will have one or more, and preferably all, of the structural characteristics or conserved features referred to below for the sequences of SEQ ID NO: 2 or SEQ ID NO: 4, preferably SEQ ID NO: 2.

Preferably, any analogs, parts or fragments as described herein will be such that they have a degree of “sequence identity”, at the amino acid level, with the amino acid sequence of SEQ ID NO: 2 or SEQ ID NO: 4 of at least 50%, preferably at least 60%, more preferably at least 70%, even more preferably at least 80%, and in particular more than 90% and up to 95% or more.

For this purpose, the percentage of “sequence identity” between a given amino acid sequence and the amino acid sequence of SEQ ID NO: 2 or SEQ ID NO: 4 may be calculated by dividing the number of amino acid residues in the given amino acid sequence that are identical to the amino acid residue at the corresponding position in the amino acid sequence of SEQ ID NO: 2 or SEQ ID NO: 4 by the total number of amino acid residues in the given amino acid sequence and multiplying by 100%, in which each deletion, insertion, substitution or addition of an amino acid residue—compared to the sequence of SEQ ID NO:2 or SEQ ID NO:4—is considered as a difference at a single amino acid (position).

Alternatively, the degree of sequence identity may be calculated using a known computer program, such as those mentioned above.

Also, such sequence identity at the amino acid level may take into account so-called “conservative amino acid substitutions”, which are well known in the art, for example from GB-A-2 357 768, WO 98/49185, WO 00/46383 and WO 01/09300; and (preferred) types or combinations of such substitutions may be selected on the basis of the pertinent teachings from the references mentioned in WO 98/49185.

Also, preferably, any analogs, parts or fragments as described herein will have a biological activity that is essentially similar to the biological activity described above for the sequences of SEQ ID NO: 2 or SEQ ID NO: 4, i.e. to a degree of at least 10%, preferably at least 50% more preferably at least 75%, and up to 90%, as measured by the assay mentioned above.

It is also within the scope of the invention to use a fusion of an amino acid sequence of the invention (as described above) with one or more further amino acid sequences, for example to provide a protein fusion. Generally, such fusions may be obtained by suitable expression of a suitable nucleotide sequence of the invention—such as a suitable fusion of a nucleotide sequence of the invention with one or more further coding sequences—in an appropriate host cell or host organism, as further described below.

One particular embodiment, such fusions may comprise an amino acid sequence of the invention fused with a reporter protein such as glutathione S-transferase (“GST”), green fluorescent protein (“GFP”), luciferase or another fluorescent protein moiety. As will be clear to the skilled person, such fusions may find particular use in expression analysis and similar methodologies.

In another embodiment, the fusion partner may be an amino acid sequence or residue that may be used in purification of the expressed amino acid sequence, for example using affinity techniques directed against said sequence or residue. Thereafter, said sequence or residue may be removed (e.g. by chemical or enzymatical cleavage) to provide the nucleotide sequence of the invention (for this purpose, the sequence or residue may optionally be linked to the amino acid sequence of the invention via a cleavable linker sequence). Some preferred, but non-limiting examples of such residues are multiple histidine residues and glutatione residues.

In one preferred, but non-limiting aspect, any such fusion will have a biological activity that is essentially similar to the biological activity described above for the sequences of SEQ ID NO: 2 or SEQ ID NO: 4, i.e. to a degree of at least 10%, preferably at least 50% more preferably at least 75%, and up to 90%, as measured by the assay mentioned above.

The nucleotide sequences and amino acid sequences of the invention may generally be characterized by the presence of one or more of the following structural characteristics or conserved features:

For the gene Aphis gossypii: SEQ ID NO: 3 is a cDNA sequence encompassing the open reading frame; SEQ ID NO: 4 is the protein encoded by SEQ ID NO: 3; and SEQ ID NO: 2 is the same protein minus the signal peptide. The Aphis mAchR protein sequence is related to other muscarinic as set forth in the table below, where the relatedness values were determined using Geneworks v 2.5 program with the following parameters: cost to open gap=50, lengthen gap=100, minimum diagonal length=4, maximum diagonal offset=125. Typically when aligning muscarinic receptor amino acid sequences, the highly variable amino acid sequence in the third intracellular loop located between transmembrane V and VI are excluded from the Identity calculations. Identity values calculated by including and excluding the third loop are set forth in the table below.

Percentage Identity

| | Excluding | Including Intracellular |
|---|---|---|
| Sequence: | Intracellular loop 3 | loop 3 |
| Drosophila | 70 | 36 |
| muscarinic¹ | | |
| Mouse m1 | 44 | 35 |
| muscarinic² | | |
| C. Elegans | 48 | 37 |
| gar-3³ | | |
| Aphis | 100 | 100 |
| gossypii | | |
| (SEQ ID | | |
| NO: 2) | | |

¹Genebank Accession No. M27495, Shapiro et al., Proc. Natl. Acad. Sci. USA 86, 9039-9043, 1989.
²Genebank Accession No. NM_007698, Shapiro et al., J. Biol. Chem. 263: 18397-18403, 1988.
³Genebank Accession No. ADD48771, Hwang et al., Receptor Channels 6: 415-424, 1999.

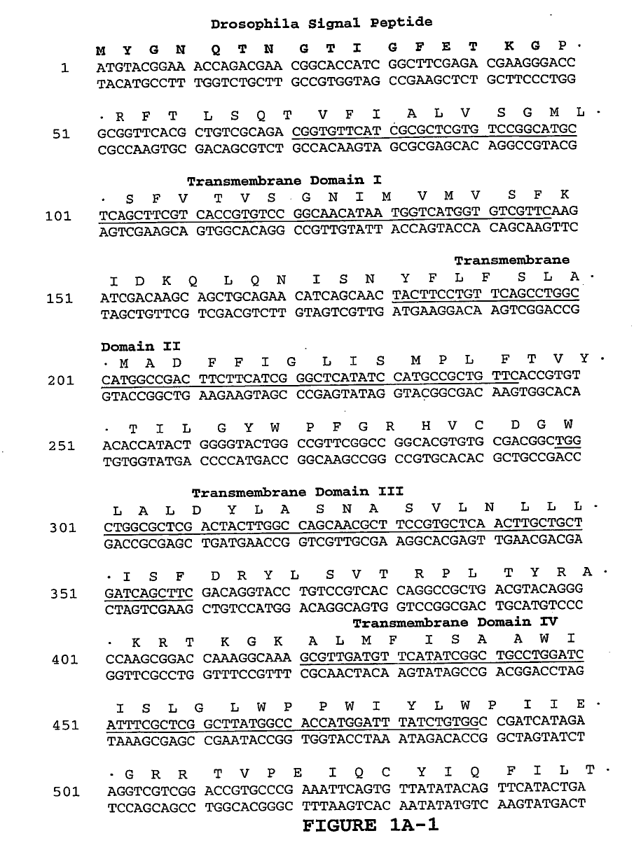

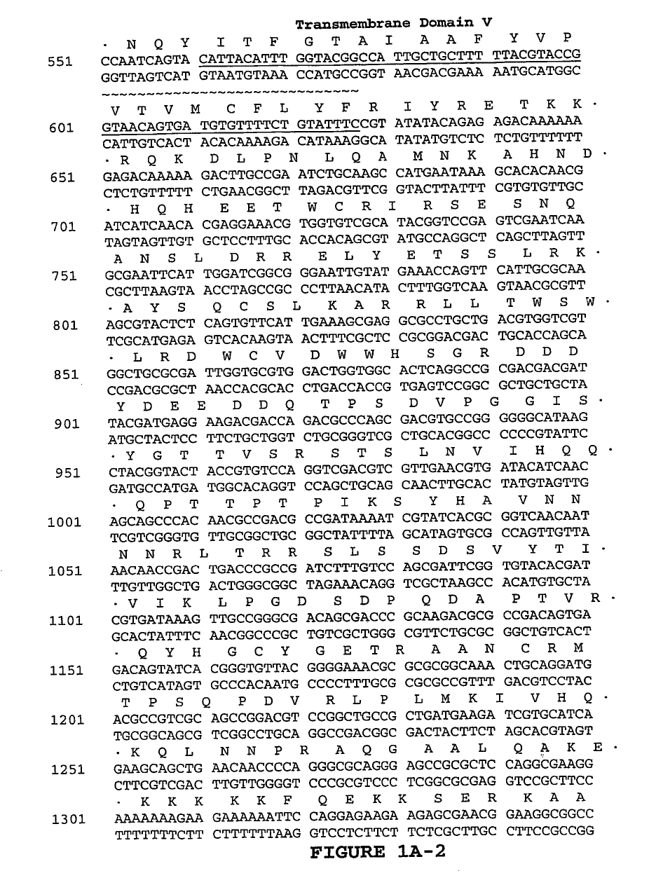

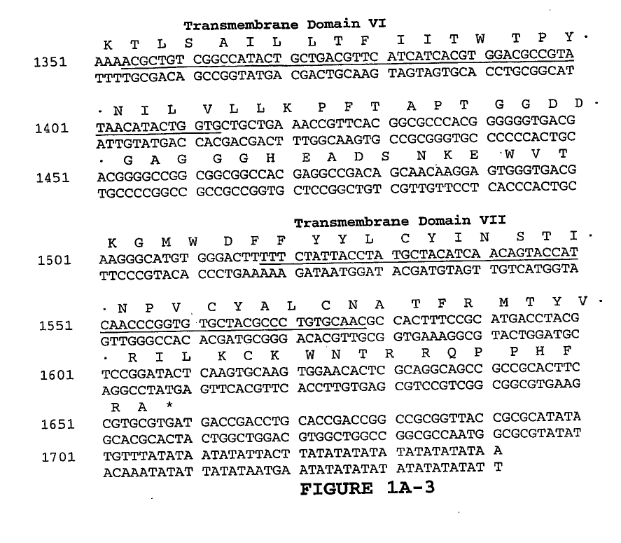

In FIG. 1A, the Drosophila appended signal peptide sequence is denoted in bold, while the remaining Aphis amino acid sequence is denoted with normal type. The present invention further relates to isolated proteins in which these signal proteins are removed or substituted with another signal sequence. The substitution of one signal sequence with another and expression of the resulting proteins is illustrated, for the echistatin protein expressed in Sf9 cells, by Daugherty et al., DNA Cell Biol. 9: 453-9, 1990. These authors also describe the use of computer-aided signal peptide selection.

Further, in FIG. 1A, the underlined seven peptide sequences in the protein encoding aphid mAchR receptor indicates the apparent transmembrane domain segments. The third intracellular located between transmembrane domain V and VI is characteristic of a muscarinic receptor and the G-protein coupling of such a receptor.

By analogy to other muscarinic receptors, it is likely that the functional protein is monomeric. See, e.g., Hannan and Hall, In Comparative Molecular Neurobiology, Y. Pichon, 1993, Birkhuaser Verlag Basel Switzerland).

On the basis of the above, and although the invention is not specifically limited to any specific explanation or mechanism, the nucleotide sequences and amino acid sequences have (biological) activity as a receptor. In particular, the present invention has shown activity as a muscarinic receptor from insects of the order Hemiptera, which are aphids, leafhoppers, whiteflies, scales and true bugs that have mouthparts adapted to piercing and sucking.

As is known in the art, biological activity of this kind can be measured using standard assay techniques, for example, through competition with a labeled, known ligand for binding sites; by measuring calcium flux using calcium fluorescent dyes; by measuring induction of inostitol phosphates using [3H]-inositol or inhibition of agonist induced stimulation of inostitol; by guanosine 5′-[γ-³⁵S]-triphosphate binding to the muscarinic receptor; through inhibition of agonist induced stimulation of guanosine 5′-[γ-³⁵S]-triphosphate binding; by fluorescent assays based on protein interactions such as fluorescence resonance energy transfer, time resolved fluorescence or fluorometic or calorimetric reporter assays; or any technology suitable for assaying Gq protein coupled receptors (see Dick et al. and Trimmer et al.). Preferably, the biological activity is measured by measuring calcium flux using calcium fluorescent dyes. However, assays to screen the muscarinic receptor from native tissue are difficult given the low expression levels in the insect CNS at about 20-150 fM mg-1 protein (see Trimmer et al.).

Another embodiment of the invention relates to a nucleic acid probe that is capable of hybridizing with a nucleotide sequence of the invention under conditions of moderate stringency, preferably under conditions of high stringency, and in particular under stringent conditions (all as described above). Such nucleotide probes may for instance be used for detecting or isolating a nucleotide sequence of the invention or as a primer for amplifying a nucleotide sequence of the invention; all using techniques known per se, for which reference is again made to the general handbooks such as Sambrook et al. and Ausubel et al., mentioned above.

Preferably, when to be used for detecting or isolating another nucleotide sequence of the invention, such a nucleotide probe will usually have a length of between 15 and 100 nucleotides, and preferably between 20 and 80 nucleotides. When used as a primer for amplification, such a nucleotide probe will have a length of between 25 and 75 nucleotides, and preferably between 20 and 40 nucleotides.

Generally, such probes can be designed by the skilled person starting from a nucleotide sequence or amino acid sequence of the invention—and in particular the sequence of SEQ ID NO: 1 or SEQ ID NO: 2—optionally using a suitable computer algorithm. Also, as will be clear to the skilled person, such probes may be degenerate probes.

In a further aspect, the invention relates to methods for preparing mutants and genetic constructs of the nucleotide sequences of the present invention.

Natural mutants of the nucleotide sequences of the present invention may be obtained in a manner essentially analogous to the method described in the Experimental Part, or alternatively by:
construction of a DNA library from the species of interest in an appropriate expression vector system, followed by direct expression of the mutant sequence;
construction of a DNA library from the species of interest in an appropriate expression vector system, followed by screening of said library with a probe of the invention (as described below) or with a nucleotide sequence of the invention;
isolation of mRNA that encodes the mutant sequence from the species of interest, followed by cDNA synthesis using reverse transcriptase;
   or by any other suitable method(s) or technique(s) known per se, for which reference is for instance made to the standard handbooks, such as Sambrook et al., “Molecular Cloning: A Laboratory Manual” (2nd.ed.), Vols. 1-3, Cold Spring Harbor Laboratory Press (1989) and F. Ausubel et al., “Current protocols in molecular biology”, Green Publishing and Wiley Interscience, New York (1987).

Techniques for generating such synthetic sequences of the nucleotide sequences of the present invention will be clear to the skilled person and may for instance include, but are not limited to, automated DNA synthesis; site-directed mutagenesis; combining two or more parts of one or more naturally occurring sequences, introduction of mutations that lead to the expression of a truncated expression product; introduction of one or more restriction sites (e.g. to create cassettes or regions that may easily be digested or ligated using suitable restriction enzymes), and the introduction of mutations by means of a PCR reaction using one or more “mismatched” primers, using for example a sequence of a naturally occurring GPCR as a template. These and other techniques will be clear to the skilled person, and reference is again made to the standard handbooks, such as Sambrook et al. and Ausubel et al., mentioned above.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors include:
vectors for expression in mammalian cells: pSVL SV40(Pharmacia)p, pMAMneo (Clontech), pcDNA3 (Invitrogen), pMC1neo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565);
vectors for expression in bacterial cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen), pEI1 (Novagen), pMTV/5His (Invitrogen).

In a further aspect, the invention relates to methods for transforming a host cell or a host organism with a nucleotide sequence, with a nucleic acid or with a genetic construct of the invention. The invention also relates to the use of a nucleotide sequence, of a nucleic acid or of a genetic construct of the invention transforming a host cell or a host organism.

According to one specific embodiment, the expression of a nucleotide sequence of the invention in a host cell or host organism may be reduced, compared to the original (e.g. native) host cell or host organism. This may for instance be achieved in a transient manner using antisense or RNA-interference techniques well known in the art, or in a constitutive manner using random, site specific or chemical mutagenesis of the nucleotide sequence of the invention.

Suitable transformation techniques will be clear to the skilled person and may depend on the intended host cell or host organism and the genetic construct to be used. Some preferred, but non-limiting examples of suitable techniques include ballistic transformation, (micro-)injection, transfection (e.g. using suitable transposons), calcium phosphate mediated transfection, electroporation and lipofection. For these and other suitable techniques, reference is again made to the handbooks and patent applications mentioned above.

After transformation, a step for detecting and selecting those host cells or host organisms that have been successfully transformed with the nucleotide sequence or genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

In yet another aspect, the invention relates to methods for producing an amino acid sequence of the invention.

To produce or obtain expression of the amino acid sequences of the invention, a transformed host cell or transformed host organism may generally be kept, maintained or cultured under conditions such that the (desired) amino acid sequence of the invention is expressed or produced. Suitable conditions will be clear to the skilled person and will usually depend upon the host cell or host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell or host organism used. Also, the amino acid sequence of the invention may be glycosylated, again depending on the host cell or host organism used.

The amino acid sequence of the invention may then be isolated from the host cell or host organism or from the medium in which said host cell or host organism was cultivated, using protein isolation and purification techniques known per se, such as (preparative) chromatography and electrophoresis techniques, differential precipitation techniques, affinity techniques (e.g. using a specific, cleavable amino acid sequence fused with the amino acid sequence of the invention) and preparative immunological techniques (i.e. using antibodies against the amino acid sequence to be isolated).

In one embodiment, the amino acid sequence thus obtained may also be used to generate antibodies specifically against said sequence or an antigenic part or epitope thereof.

In one embodiment, the present invention relates to antibodies, for example monoclonal and polyclonal antibodies, that are generated specifically against amino acid sequences of the present invention, preferably SEQ ID NO: 2, or an analog, variant, allele, ortholog, part, fragment or epitope thereof. In another embodiment, the present invention relates to antibodies, for example monoclonal and polyclonal antibodies, that are generated specifically against amino acid sequences of the present invention, preferably SEQ ID NO: 4, or an analog, variant, allele, ortholog, part, fragment or epitope thereof.

Such antibodies, which form a further aspect of the invention, may be generated in a manner known per se, for example as described in GB-A-2 357 768, U.S. Pat. No. 5,693,492, WO 95/32734, WO 96/23882, WO 98/02456, WO 98/41633 and WO 98/49306. Often, but not exclusively, such methods will involve as immunizing a immunocompetent host with the pertinent amino acid sequence of the invention or an immungenic part thereof (such as a specific epitope), in amount(s) and according to a regimen such that antibodies against said amino acid sequence are raised, and than harvesting the antibodies thus generated, e.g. from blood or serum derived from said host.

For instance, polyclonal antibodies can be obtained by immunizing a suitable host such as a goat, rabbit, sheep, rat, pig or mouse with (an epitope of) an amino acid sequence of the invention, optionally with the use of an immunogenic carrier (such as bovine serum albumin or keyhole limpet hemocyanin) or an adjuvant such as Freund's, saponin, aluminium hydroxide or a similar mineral gel, or keyhole limpet hemocyanin or a similar surface active substance. After a suitable immune response has been raised (usually within 1-7 days), the antibodies can be isolated from blood or serum taken from the immunized animal in a manner known per se, which optionally may involve a step of screening for an antibody with desired properties (i.e. specificity) using known immunoassay techniques, for which reference is again made to for instance WO 96/23882.

Monoclonal antibodies may for example be produced using continuous cell lines in culture, including hybridoma-based and similar techniques, again essentially as described in the above cited references. Accordingly, cells and cell lines that produce monoclonal antibodies against an amino acid sequence of the invention form a further aspect of the invention, as do methods for producing antibodies against amino acid sequences of the invention, which methods may generally involve cultivating such a cell and isolating the antibodies from the culture or medium, again using techniques known per se.

Also, Fab-fragments against the amino acid sequences of the invention (such as F(ab)₂, Fab′ and Fab fragments) may be obtained by digestion of an antibody with pepsin or another protease, reducing disulfide-linkages and treatment with papain and a reducing agent, respectively. Fab-expression libraries may for instance be obtained by the method of Huse et al., 1989, Science 245:1275-1281.

In another embodiment, the amino acid sequence of the invention, or a host cell or host organism that expresses such an amino acid sequence, may also be used to identify or develop compounds or other factors that can modulate the (biological) activity of, or that can otherwise interact with, the amino acid sequences of the invention, and such uses form further aspects of the invention. As will be clear to the skilled person, in this context, the amino acid sequence of the invention will serve as a target for interaction with such a compound or factor.

In this context, the terms “modulate”, “modulation, “modulator” and “target” will have their usual meaning in the art, for which reference is inter alia made to the definitions given in WO 98/06737. Generally, a modulator is a compound or factor that can enhance, inhibit or reduce or otherwise alter, influence or affect (collectively referred to as “modulation”) a functional property of a biological activity or process (for example, the biological activity of an amino acid sequence of the invention).

In this context, the amino acid sequence of the invention may serve as a target for modulation in vitro (e.g. as part of an assay or screen) or for modulation in vivo (e.g. for modulation by a compound or factor that is known to modulate the target, which compound or factor may for example be used as an active compound for agrochemical, veterinary or pharmaceutical use).

For example, the amino acid sequences, host cells or host organisms of the invention may be used as part of an assay or screen that may be used to identify or develop modulators of the amino acid sequence of the invention, such as a primary screen (e.g. a screen used to identify modulators of the target from a set or library of test chemicals with unknown activity with respect to the target) or a secondary assay (e.g. an assay used for validating hits from a primary screen or used in optimizing hit molecules, e.g. as part of hits-to-leads chemistry).

For instance, such an assay or screen may be configured as an in vitro assay or screen, which will generally involve binding of the compound or factor to be tested as a potential modulator for the target (herein below also referred to as “test chemical”) to the target, upon which a signal generated by said binding is measured. Suitable techniques for such in vitro screening will be clear to the skilled person, and are for example described in Eldefrawi et al., (1987). FASEB J., Vol. 1, pages 262-271 and Rauh et al., (1990), Trends in Pharmacol. Sci., vol. 11, pages 325-329. For example, such an assay or screen may be configured as a binding assay or screen, in which the test chemical is used to displace a detectable ligand from the target (e.g. a radioactive or fluorescent ligand), upon which the amount of ligand displaced from the target by the modulator is determined.

Such an assay or screen may also be configured as a cell-based assay or screen, in which a host cell of the invention is contacted with or exposed to a test chemical, upon which at least one biological response by the host cell is measured.

Also, such an assay or screen may also be configured as an whole animal screen, in which a host organism of the invention is contacted with or exposed to a test chemical, upon which at least one biological response (such as a phenotypical, behavioral or physiological change, including but not limited to paralysis or death) by the host organism is measured.

Thus, generally, the assays and screens described above will comprise at least one step in which the test chemical is contacted with the target (or with a host cell or host organism that expresses the target), and in particular in such a way that a signal is generated that is representative for the modulation of the target by the test chemical. In a further step, said signal may then be detected.

Accordingly, in one aspect, the invention relates to a method for generating a signal that is representative for the interaction of an amino acid sequence of the invention with a test chemical, said method at least comprising the steps of:
a) contacting the amino acid sequence of the invention, or a host cell or host organism containing or expressing an amino acid sequence, with said test chemical, in such a way that a signal may be generated that is representative for the interaction between said test chemical and said amino acid sequence; and optionally
b) detecting the signal that may thus be generated.

In another aspect, the invention relates to a method for identifying modulators of an amino acid sequence of the invention (e.g. from a set or library of test chemicals), said method at least comprising the steps of:
a) contacting the amino acid sequence of the invention, or a host cell or host organism containing or expressing an amino acid sequence, with a test chemical, in such a way that a signal may be generated that is representative for the interaction between said test chemical and said the target; and optionally
b) detecting the signal that may thus be generated, said signal identifying the modulator of said amino acid sequence.

Accordingly, the present invention provides methods of identifying a modulator of a hemipteran muscarinic receptor protein activity. The methods comprise the step of performing a test assay by contacting a test cell, which comprises a recombinant expression vector that contains a nucleic acid sequence that encodes the hemipteran muscarinic receptor and that expresses the hemipteran muscarinic receptor with a solution containing calcium in the presence of a test compound, and detecting the amount of intracellular calcium in the test cell. The methods also comprise the step performing a negative control assay by contacting a negative control cell which comprises a recombinant expression vector that contains a nucleic acid sequence that encodes the hemipteran muscarinic receptor and expresses hemipteran muscarinic receptor, with a solution containing calcium in the absence of the test compound, and detecting the amount of intracellular calcium in the negative control cell. The amount of intracellular calcium in the test cell is compared to the amount of intracellular calcium in the negative control cell. A change in the amount of and a hemipteran muscarinic receptor agonist calcium in the test cell compared to the amount of intracellular calcium in the negative control cell indicates the test compound is a modulator of a hemipteran muscarinic receptor protein activity. In some preferred embodiments, the test cell is a CHO-K1 cell. In some preferred embodiments, the intracellular calcium is detected by using an assay in which fluorescence generated by dye inside of said cell interacting with intracellular calcium is measured. In some preferred embodiments, the methods further comprise performing a positive control assay by contacting a positive control cell which comprises a recombinant expression vector that contains a nucleic acid sequence that encodes the hemipteran muscarinic receptor and expresses hemipteran muscarinic receptor, with a solution containing calcium in the absence of the test compound and in the presence of a hemipteran muscarinic receptor agonist, and detecting the amount of intracellular calcium in said positive control cell. The hemipteran muscarinic receptor agonist is preferably carbamoylcholine. In some embodiments, the methods further comprise the step of

either performing a second-type negative control assay by contacting a hemipteran muscarinic receptor negative control cell that does not express hemipteran muscarinic receptor with a solution containing calcium in the absence of the test compound, and detecting the amount of calcium taken up by said hemipteran muscarinic receptor negative control cell; and/or performing a third-type negative control assay by contacting a hemipteran muscarinic receptor negative control cell that does not express hemipteran muscarinic receptor with a solution containing calcium in the absence of the test compound and in the presence of a hemipteran muscarinic receptor agonist, and detecting the amount of calcium taken up by said hemipteran muscarinic receptor negative control cell. The hemipteran muscarinic receptor agonist is preferably carbamoylcholine. In preferred embodiments, the hemipteran muscarinic receptor protein used in the methods has an amino acid sequence selected from the group consisting of: SEQ ID NO: 2, a mutant thereof, a fragment thereof, SEQ ID NO: 4, a mutant thereof, and a fragment thereof. In some embodiments, the nucleic acid sequence that encodes the hemipteran muscarinic receptor is SEQ ID NO: 1 or SEQ ID NO: 3.

According to some embodiments, methods of identifying an inhibitor of a hemipteran muscarinic receptor protein activity comprise the step of performing a test assay by contacting a test cell, which comprises a recombinant expression vector that contains a nucleic acid sequence that encodes the hemipteran muscarinic receptor and expresses hemipteran muscarinic receptor, with a solution containing calcium and a hemipteran muscarinic receptor agonist in the presence of a test compound, and detecting the amount of intracellular calcium in said test cell. A control assay is also performed by contacting a negative control cell, which comprises a recombinant expression vector that contains a nucleic acid sequence that encodes the hemipteran muscarinic receptor and expresses hemipteran muscarinic receptor, with a solution containing calcium and a hemipteran muscarinic receptor agonist in the absence of the test compound, and detecting the amount of intracellular calcium in said negative control cell. The amount of intracellular calcium in the test cell is compared to the amount of intracellular calcium in the control cell. A decrease in the amount of intracellular calcium in the test cell compared to the amount of intracellular calcium in the control cell indicates the test compound is an inhibitor of hemipteran muscarinic receptor protein activity. In some preferred embodiments, the test cell is a CHO-K1 cell. In some preferred embodiments, the intracellular calcium is detected by using an assay in which fluorescence generated by dye inside of the cell interacting with intracellular calcium is measured. In some preferred embodiments the hemipteran muscarinic receptor agonist is carbamoylcholine. In some preferred embodiments, the hemipteran muscarinic receptor protein has an amino acid sequence selected from the group consisting of: SEQ ID NO: 2, a mutant thereof, a fragment thereof, SEQ ID NO: 4, a mutant thereof, and a fragment thereof. In some embodiments, the nucleic acid sequence that encodes said hemipteran muscarinic receptor is SEQ ID NO: 1 or SEQ ID NO: 3.

A test chemical may be part of a set or library of compounds, which may be a diverse set or library or a focused set or library, as will be clear to the skilled person. The libraries that may be used for such screening can be prepared using combinatorial chemical processes known in the art or conventional means for chemical synthesis.

The assays and screens of the invention may be carried out at medium throughput to high throughput, for example in an automated fashion using suitable robotics. In particular, in this embodiment, the method of the invention may be carried out by contacting the target with the test compound in a well of a multi-well plate, such as a standard 24, 96, 384, 1536 or 3456 well plate.

Usually, in a screen or assay of the invention, for each measurement, the target or host cell or host organism will be contacted with only a single test compound. However, it is also within the scope of the invention to contact the target with two or more test compounds—either simultaneously or sequentially—for example to determine whether said combination provides a synergistic effect.

Once a test chemical has been identified as a modulator for an amino acid sequence of the invention (e.g. by means of a screen or assay as described hereinabove), it may be used per se as a modulator of the amino relevant amino acid sequence of the invention (e.g. as an active substance for agrochemical, veterinary or pharmaceutical use), or it may optionally be further optimized for final use, e.g. to improve properties such as solubility, adsorption, bio-availability, toxicity, stability, persistence, environmental impact, etc. It will be clear to the skilled person that the nucleotide sequences, amino acid sequences, host cells or host organisms and methods of the invention may find further use in such optimization methodology, for example as (part of) secondary assays.

The invention is not particularly limited to any specific manner or mechanism in or via which the modulator (e.g. the test chemical, compound or factor) modulates, or interacts with, the target (in vivo or in vitro). For example, the modulator may an agonist, an antagonist, an inverse agonist, a partial agonist, a competitive inhibitor, a non-competitive inhibitor, a cofactor, an allosteric inhibitor or other allosteric factor for the target, or may be a compound or factor that enhances or reduces binding of target to another biological component associated with its (biological) activity, such as another protein or polypeptide, a receptor, or a part of organelle of a cell. As such, the modulator may bind with the target (at the active site, at an allosteric site, at a binding domain or at another site on the target, e.g. covalently or via hydrogen bonding), block the active site of the target (in a reversible, irreversible or competitive manner), block a binding domain of the target (in a reversible, irreversible or competitive manner), or influence or change the conformation of the target.

As such, the test chemical or modulator may for instance be:
an analog of a known substrate of the target;
an oligopeptide, e.g. comprising between 2 and 20, preferably between 3 and 15 amino acid residues;
an antisense or double stranded RNA molecule;
a protein, polypeptide;
a cofactor or an analog of a cofactor.

The test chemical or modulator may also be a reference compound or factor, which may be a compound that is known to modulate or otherwise interact with the target (e.g. a known substrate or inhibitor for the target) or a compound or factor that is generally known compound that is known to modulate or otherwise interact with other members from the general class to which the target belongs (e.g. a known substrate or inhibitor of said class).

Preferably, however, the test chemical or modulator is a small molecule, by which is meant a molecular entity with a molecular weight of less than 1500, preferably less than 1000. This may for example be an organic, inorganic or organometallic molecule, which may also be in the form or a suitable salt, such as a water-soluble salt. The term “small molecule” also covers complexes, chelates and similar molecular entities, as long as their (total) molecular weight is in the range indicated above.

The invention will now be further illustrated by means of the following non-limiting Experimental Part.

### EXPERIMENTAL PART

### Example 1

### Muscarinic Receptor Sequence Identifications

Materials and Methods.

Aphid polyA RNA isolation. A 0.1% solution of diethyl pyrocarbonate (“DEPC” available from Aldrich Chemical Co., Inc. Milwaukee, Wis.) in water was incubated at 37° C. for about 16 hours and then autoclaved for 60 minutes. All glassware was baked for four hours at 250° C. and all bottle caps were soaked in the 0.1% DEPC solution. The microprobe of a Braun homogenizer (available from B. Braun Biotech International, Allentown, Pa.) was soaked in 50 mls of 100% ethanol (available from J.T. Baker Inc., Phillipsburg, N.J.) and then run in 25 mls RNAzo1B (a guanidinium hydrochloride preparation from CINNA-BIOTECX Labs, Inc., Houston, Tex.). Cotton aphids were collected from cotton plants and placed on ice in tared centrifuge tubes. After harvesting approximately 1.2 grams of aphid material, the aphids were frozen at −70° C. The aphids (1.0 gram) were then weighed out in two 0.5 gram aliquots and were homogenized at full speed for 30 seconds at ambient temperature in a 1.5 mls of Extraction Buffer from a Pharmacia Biotech QuickPrep Micro mRNA Purification kit (herein referred to as “PMK”, available from Pharmacia Biotech Inc.). Three mls of Elution Buffer from the PMK were added and the resulting mixture was homogenized for ten seconds. The resulting macerate was clarified by centrifugation at 12000 g in an SS34 rotor (available from Sorvall Products, L.P, Asheville, N.C.) for 10 minutes at ambient temperature. The supernatant was batch processed on oligo-dT spin columns from the PMK as specified by the PMK. Three elutions totaling 1.5 ml for each of two columns were pooled and the RNA quantified by UV spectrometry. The mRNA was precipitated in by adding 130 uls of potassium acetate coultion, 2.6 mls of absolute ethanol and 20 uls of glycogen provided in the PMK. The tubes were stored at 20° C. for about sixteen hours and the resulting mRNA precipitate was pelleted by centrifugation at 14000 g for 15 minutes. The pellets were washed with an 80% aqueous ethanol solution and resuspended in distilled water.

Synthesis of first strand cDNA. Reverse transcription was initiated by addition of Avian Myeloblastosis Virus (“AMV”) reverse transcriptase I (Promega Corp., Madison, Wis.) to 1.0 μg of template RNA. The reverse transcription reaction also included the following reagents contained in the Promega Universal RiboClone cDNA Synthesis Kit: 5 uls of First Strand Reaction Buffer, 1 uls of Recombinant RNasin Ribonuclease Inhibitor, 2.5 uls of 40 mM Sodium Pyrophosphate and either Random Hexamers, Oligo(dT)15 primers, or degenerate gene specific primers. For 3′ RACE reactions, the mRNA was reverse transcribed with using a GIBCO Oligo(dT) Adapter Primer (AP) (available from GIBCO-BRL). For 5′ Race reactions gene specific primers were used for the reverse transcription. The reactions were placed on a Geneamp9600 thermal cycler (Perkin-Elmer-ABI) and held for 60 minutes at 37° C. for Random Hexamers, 42° C. for Oligo(dT)15 and Gibco Oligo(dT) Adapter primers, or 50° C. for gene specific primers; the AMV RT was then inactivated by heating to 99° C. for 5 minutes followed by 5 minutes at 5° C.

Synthesis of second strand cDNA. Second strand synthesis was conducted according to the method of Gubler and Hoffman (Gene, 25, 263, 1983) using the Marathon cDNA Amplification Kit from Clontech (Clontech Laboratories, Palo Alto, Calif.). Double stranded cDNA was synthesized by taking the first strand reactions and adding Second Strand Reaction Buffer, all four dinucleotides in a 10 mM mix, DNA polymerase and Rnase H. The reactions were incubated for 2 hours at 14° C. on a Geneamp 9600 thermal cycler completing the second strand synthesis. Ten units of T4 DNA polymerase from the Marathon cDNA Amplification Kit were added. The resulting cDNA was incubated at 16° C. for 45 minutes to create blunt ends on the cDNA. The reactions were terminated with ethylenediaminetetraacetic acid (EDTA, available from Aldrich Chemical Co.) and ds cDNA extracted by phenol chloroform as described in Maniatis et al. (Molecular Cloning, a Laboratory Manual, Cold Spring Harbor Press, 1989). The aqueous phase containing ds cDNA was ethanol precipitated with one-half volume of 4M ammonium acetate and 2.5 volumes of 100% ethanol. The resulting ds cDNA pellet was washed with an 80% aqueous ethanol solution and resuspended in distilled water. For 5′ RACE reactions, the ds cDNA was ligated with the Clontech Marathon cDNA adapter using T4 DNA ligase.

PCR amplification. The 20 μl cDNA reaction was made to 100 μl utilizing buffers and dNTPs supplied in a Perkin Elmer, Amplitaq based RT-PCR kit according to Perkin Elmer's protocol. Amplifications utilizing degenerate primers typically employed annealing temperatures in the range of 45 to 55° C., those involving isoform specific primers used annealing temperatures in the range of 50 to 60° C. In both instances, touchdown PCR was utilized in which the annealing temperature was decreased one half a degree per cycle for 20 cycles or 10 degrees. 3′ and 5′ RACE reactions were carried out using primers and protocols supplied with a GIBCO-BRL 3′ RACE kits (GIBCO-BRL) and the Clontech Marathon cDNA Amplification Kit, respectively. The PCR products were characterized by agarose gel electrophoresis. When secondary “nested” amplifications were carried out, bands were excised from NuSieve gels (FMC Corp., Philadelphia, Pa.) and melted by heating to 70° C. The molten agarose was diluted 1:1 with warm water and a 1:5 μl aliquot was transferred directly to a second 100 μl amplification vessel.

Primer synthesis and design. Oligonucleotides were synthesized by Life Technologies Inc. and provided as lyophilized pellets which were reconstituted in 1× PCR Reaction buffer from Perkin Elmer prior to use. PCR primers and probes were designed and annealing temperatures estimated using the OLIGO 4.0 program from NBI Scientific Software (Plymouth, Minn.) and Consensus-Degenerate Hybrid Oligonucleotide Primers Software (“CODEHOP”) from Henikoff et al. (Nucleic Acids Research, 26, 70, 1628-1635, 1998) available on line through the Fred Hutchinson Cancer Research Center.

Subcloning and sequencing. Proteins were removed from PCR reactions by three extractions with Strataclean resin as specified by Stratagene Corp. (La Jolla, Calif.). If the primers included engineered restriction sites, they were then digested. More routinely, the amplimers were blunt ended by filling with Klenow polymerase treatment, then phosphorylated by routine procedures (Sambrook et al., 1989). The amplimers were then gel purified on Seaplaque or NuSieve gels (FMC Corp.) and extracted from the agarose using a QIAEX kit (available from QIAGEN Corp., Chatsworth, Calif.). Alkaline lysis plasmid isolations and purifications were carried out with a Qiaquik kit (available from QIAGEN Corp.) following the recommendations of QIAGEN Corp. Thermal cycle sequencing reactions utilized dye terminator cycling reactions were carried out with a Perkin Elmer Amplitaq FS sequencing kit (available from Perkin-Elmer-ABI) and the reaction products were analyzed on 5% Long Ranger gels run in an ABI Prizm 377 automated DNA sequencer (available from Perkin-Elmer-ABI). Five to ten clones carrying a PCR reaction product were sequenced in both directions until a consensus could be achieved between multiple clones as a means of avoiding errors in nucleotide assignments due to thermal polymerase mis-incorporations. Sequencing contigs were assembled using the Intelligenetics GeneWorks program.

Primers. The primers utilized were as follows:

| | | | |
|---|---|---|---|
| Primer | Sequence | Translation | Orientation |

| | | | |
|---|---|---|---|
| 1 (#5) | GGCAACGTGATGGTGATGATHWSNTTYAA | GNVMVMISFK | forward |
| | n-terminus nested | deg | |
| 2 (#6) | GGTGGGTCTCGATGAACTGDATRTARCA | CYIQFIETNQ | reverse |
| | n-terminus nested | Deg | |
| 3 (#7) | CCGGTTTCTCGCCAGATNCKCCARTA | YWRIWRET | reverse |
| | n-terminus reverse trans | Deg | |
| 4 (#8) | GGCAACGTGATGGTGATGATHWSNTTYAA | GNVMVMISFK | forward |
| | n-terminus first pcr | Deg | |
| 5 (#9) | TAGAAAGCAGCCATAGCGTNCCRAANGT | TFGTAMAAFY | reverse |
| | n-terminus first pcr | Deg | |
| 6 (#10) | CCGGCGGACGAGTGYTAYATHCARTT | PAHECYIQ | forward |
| | il loop to tm6 nested | Deg | |
| 7 (#11) | TGGGGTTGATGGTGGAGTTNAYRTARCANA | CYINSTINPM | reverse |
| | il loop to tm6 nested | Deg | |
| 8 (#12) | CTCCGGAAGGTTGCGTTGANARNGCRTA | YALCNTAF | reverse |
| | il loop first par | Deg | |
| 9 (#13) | CGAGCGAAATGATCCAGG | AWIISL | reverse |
| | rt for 5′ race | | |
| 10 (#14) | AGGTACCTGTCGAAGCTGATCAG | ISFDRY | reverse |
| | first pcr 5′ race | | |
| 11 | CCATCCTAATACGACTCACTATAGGGC | | forward |
| | adaptor primer 1 Clontech | | |
| 12 (#15) | AGCACGGAAGCGTTGC | SNASVL | reverse |
| | nest pcr 5′ race | | |
| 13 | ACTCACTATAGGGCTCGAGCGGC | | reverse |
| | adaptor primer 2 Clontech | | |
| 14 (#16) | CGACAGCAACAAGGAGTGG | DSNKEW | forward |
| | 3′ race | | |
| 15 | CUACUACUACUAGGCCACGCGTCGACTAGTAC | | reverse |
| | Gibco 3′ Race universal adaptor primer | | |

^{♦}The SEQ ID NO: numbers are in parentheses.

The degenerate primers among the above oligonucleotides incorporate a statistical mix of monomers at the positions labeled N (A, G, C or T), H (A, C or T), S (C or G), Y (C or T), W (A or T), D (A, G or T) or R (A or G) [in accordance with IUPAC convention]. The underlined sequences are restriction sites.

Aphis gossypii—Sequence Amplifications

All amplification descriptions for the Aphis gossypii designate sequence positions with respect to the corresponding sequences set forth in FIG. 1A. Transmembrane domains are underlined.

In a nested PCR reaction, first Primers 1 and 5, and then Primers 1 and 2 were used to amplify a fragment from nucleotides 121 to 558 Aphis gossypii sequence, which was cloned and sequenced. This region is comprised of sequence just upstream of transmembrane domain 2 to transmembrane domain 5 as shown in FIG. 1A. It will be recognized, for this amplification and in the other amplifications from mRNA described herein, that the amplification substrate was produced by reverse transcription with a reverse primer, in this case with Primer 3.

The Aphis sequence was extended upstream through transmembrane domain 1 by reverse transcribing mRNA with primer 9, followed by 5′ RACE utilizing the Clontech Marathon cDNA kit. Touchdown PCR with primers 10 and 11 was followed by nested PCR with primers 12 and 13 utilizing cDNA generated from the reaction with primers 10 and 11. The resulting amplimer generated sequence information from nucliotides 49 to 338. Primer 11 and 13 are Clontech Adaptor Primers 1 and 2, respectively, from the Marathon cDNA kit.

The Aphis sequence comprising the regions just upstream of transmembrane domain 5 through transmembrane domain 7, including the large intracellular loop 3, was generated by reverse transcription with random hexamers from a Promega Riboclone Kit (available from Promega Corp.), followed by touchdown PCR with primers 6 and 8. The cDNA generated from this reaction was then amplified with the nested primers 6 and 7 generating an amplimer from 516 to 1556. Finally, the 3′ end was generated by reverse transcription with random hexamers followed by PCR with primer 14 and 15. Primer 15 is the Universal adaptor Primer (“UAP”) provided in the GIBCO-BRL 3′-RACE system. An amplimer from 1426 through the stop codon at 1657-1659 was generated. The amplimers from the above reactions were isolated, cloned, and sequenced.

Drosophila Sequence Amplifications

In order to add a translational start site to the Aphis sequence, a chimera was constructed by adding the Drosophila signal peptide for the muscarinic receptor to the 5′ end of the Aphis sequence. The Drosophila signal peptide was prepared in a two-step approach whereby it was first isolated using primers that exactly matched its sequence, and then modified using engineered primers. Drosophila larvae were purchased from Carolina Biolgical Supply (Burlington, N.C.) and extracted for mRNA in the same manner as set forth above for the aphids.

Drosophila mRNA was reverse transcribed with random hexamers, then PCR reactions were conducted first between Primers 17 and 16 followed by a nested reaction between Primers 17 and 18, yielding a fragment from 73 to 311 of the Drosophila 5′ end using numbering in Genbank deposit M27495 including a portion of the 5′UTR, start codon, and signal peptide. The resulting amplimer was modified by reamplifying with primers 19 and 20 to add appropriate restriction sites, SmaI half-site and SacII for 19, and SacI, XhoI, and BamHI for 20, for ligation to the aphid sequence. The resulting amplimer yielded a fragment from positions 63 to 224 of the Drosophila sequence using the numbering in Genbank depsoit M27495. The amplimer was polished, phosphorylated, ligated into SmaI cut pUC18 (available as “Ready to Go pUC18” from Pharmacia Biotech Inc.), fully sequenced and found to be in complete agreement with the GenBank deposit (using the numbering of GenBank accession M27495). The plasmid was labeled pUCDros.

### Example 2

### Expression Vectors

Primers. The primers utilized were as follows: (underlined sequence indicates restriction sites added)

| | | | |
|---|---|---|---|
| Primer | Sequence | Translation | Orientation |

| | | | |
|---|---|---|---|
| 16 (#17) | ccagcgagaacaagaagtagttg | SNYFLFSLA | reverse |
| 17 (1#18) | cagggtattcgccaggatac | 5′ UTR | forward |
| 18 (1#19) | tcatcaccatgacattgcc | GNVMVM | reverse |
| 19 (1#20) | gggagtgagtaccgcggtcccttc | TKGPRYSL | reverse |
| 20 (1#21) | Agcgagctcgaggatccctattccaccatgtacgg | 5′ UTR-MYG | forward |
| 21 (1#22) | cagcccaggttcacgctgtcg | QPRFTLS | forward |
| 22 (1#23) | gcgcaatgaactggtttca | ETSSLRK | reverse |
| 23 (1#24) | cgtgttgatgatcgttgtgtg | ESNQANS | reverse |
| 24 (1#25) | ggtcggtgcaggtcggtc | 3′ UTR | reverse |
| 25 (1#26) | ctgtgatgttcatatcggctg | LMFISAA | forward |
| 26 (1#27) | cacgcacggaagtgcggc | PHFRA* | reverse |
| 27 (1#28) | tacgtaccggtaacagtgatgtg | YVPVTVMC | forward |
| 28 (1#29) | Gtcgaagcttgaggattccctgcggtcatcacgcac | HFRA*- | reverse |
| | ggaagtg | 3′ UTR | |

Aphis Muscarinic Receptor

Two overlapping pieces of Aphis muscarinic receptor were amplified, and a unique EcoRI site was used to ligate the two pieces into a complete Aphis muscarinic sequence. The 3′ piece of this unique EcoRI site was created using Primer 22 to prime reverse transcription (beginning at 799 in FIG. 1A), followed by PCR with Primers 21 and 22, nested with primers 23 and 21. The 5′ piece containing the unique EcoRI site was created using Primer 24 to prime reverse transcription (beginning at 378 in FIG. 1A), followed by nested PCR with first, Primers 25 and 26, and then Primers 27 and 26. Restriction sites were added by reamplifying the amplimer with Primers 27 and 28. Both amplimers were polished, phosphorylated, and ligated into a pUC18 and sequenced.

To assemble the complete sequence for expression, the vector pUCDros containing the Drosophila signal peptide amplimer as described above was ligated to the 5′ Aphis sequence generated as described above by digesting the vector, pUC18Dros, with SacII and SmaI (available from New England Biolabs, Beverly, Mass.) and then dephosphorylating the ends. The 5′ Aphis sequence generated as described above from primers 21 and 23 was polished, phosphorylated, digested with SacII and then ligated into the pUC vector containing the Drosophila signal peptide, pUC18Dros. The resulting vector was sequenced and labeled pUC18DrosAphid21-23.

The complete Aphis sequence was then generated by digesting the vector containing the Drosophila signal peptide and the 5′ Aphis sequence, pUC18DrosAphid21-23, with EcoRI to release the fragment containing the Drosophila signal peptide and 5′ Aphis fragment. The resulting fragment was then inserted into the vector containing the 3′ end of the Aphis sequence, pUC18Aphid27-28, by first digesting the vector with EcoRI and dephosphorylating the ends. The 5′ fragment was then ligated into the EcoRI digested pUC18Aphid27-28 vector completing assembly of the gene. The insert from this vector, pUC18DrosAphidmAchR, was sequenced and used to provide an insert for the expression vector.

To construct the expression vector, pSVL SV40 Late Promoter Expression Vector, with Genebank Accession Number U13868, was used to constitutively express the aphid protein. To assemble the expression vector, the pSVL 40 Late Promoter Expression Vector was digested with BamHI (available from New England Biolabs) and dephosphorylated. The complete Drosophila Aphis muscarinic chimeric fragment was released from the pUC18DrosAphidmAchR vector by similarly digesting with BamHI. The resulting fragment was ligated into the pSVL SV40 vector and sequenced. The resulting expression vector was labeled pSVLmAchR6-10.

### Example 3

### Muscarinic Receptor Stable Cell Line

Materials and Methods.

The expression vector, pSVLmAchr 6-10, was used to express the aphid muscarinic receptor in CHO cells. The pSVLmAchR 6-10 vector was transformed into CHO K1 cells obtained from American Type Tissue Culture (“ATTC”, Manassa, Va.) using the calcium phosphate method as outlined in John Wiley and Sons, Inc. (Current Protocols in Molecular Biology, 2000) and Jarvis et al. (Methods in Molecular Biology, ed. Richardson, 1995, Humana Press Inc. NJ, Vol. 39 187-201). To generate stable cell lines, the vector pNeo Dominant Selectable Marker Vector (available from Pharmacia Biotech Inc.), with Genebank Accession Number U13862, was cotransfected with the pSVLmAchR 6-10 vector to confer resistance to the antibiotic, geneticin or G-418. The pNeo plasmid codes for the protein aminoglycoside 3′-phosphotransferase (“APH”), which can metabolize the antibiotic G-418. When the APH protein is expressed in the cells, the cells become resistant to the antibiotic G-418. Furthermore, cotransfecting the CHO-K1 cells with the pNeo and pSVLmAchR6-10 vectors allows for selection of cells expressing both the APH protein and the Aphis muscarinic receptor with the expectation that approximately fifty percent of the cells containing the pNeo plasmid can also be expected to also contain the psvlmachr plasmid as described by Jarvis et al. (Methods in Molecular Biology, ed. Richardson, 1995, Humana Press Inc. Vol. 39 198).

After transfecting the CHO-K1 cells for 3-16 hours in a calcium phosphate solution containing the pSVLmAchR 6-2 and pNeo vectors, the cells were rinsed with fresh media F-12K Nutrient Mixture Media and supplemented with 10% Fetal Bovine Serum (both available from GIBCO-BRL) without antibiotic for about 16 hours. Transformed cells were then selected by adding 800 mg/ml of the G-418 to the media and incubating the cells at 37° C. in 5% carbon dioxide. After 7 to 10 days isolated colonies were apparent and at 14 days cells were cloned by removing individual colonies using cloning cylinders as described in known protocols as described by Jarvis et al. (Methods in Molecular Biology, ed. Richardson, 1995, Humana Press Inc., Vol. 39, 198.). Control cells which were not transformed with pNeo perished after this exposure. Clonal colonies were then grown to near confluence in 96 well tissue culture plates with constant exposure to G-418 at 880 mg/ml. In order to ensure monoclonal clones, the cells from the 96 well plates were diluted, plated on 60 mm tissue culture petri plates and then clones were, selected after about 10-14 days growth. The clones were then picked once again using cloning cylinders and placed in 96 well plates. Clones were grown up successively in 48, 24, and 6 well plates under selection with G-418 at 880 mg/ml. Clones were then transferred to 25 mls flasks and passaged in F-12K media containing the antibiotic G-418 at 880 mg/ml. The passaged clones were screened for muscarinic activity by monitoring calcium efflux induced by the addition of the muscarinic agonist carbamoylchloline chloride (available from Sigma-Aldrich Co.). Calcium flux was measured on a FLIPR® 384 Fluorometric Imaging Plate Reader system (available from Molecular Devices, Sunnyvale Calif.), set up in a 96 well plate format using a FLIPR® Calcium No Wash Assay Dye Kit (available from Molecular Devices) as per the manufacturer's directions for measuring calcium in CHO-K1 cells. Clones were selected by their potency or amplitude of signal generated in the presence of the agonist carbamoylchloline chloride and their efficacy in terms of dose of agonist required to achieve 50% of the maximal fluorescence induced by 1 mM carbamoylchloline chloride. Clone pSVLmAchR 6-2 was chosen for the development of the assay set forth in Example 4 below.

### Example 4

### Assay Method

Assays were performed using CHO-K1 cells stably transfected with the pSVLmAchR and pNeo vector described above and identified as clone pSVLmAchR 6-2. Cells were grown at 37° C. in 5% carbon dioxide in F-12K medium containing 10% fetal calf serum (available from GIBCO-BRL). Cells were removed from the culture flask using EDTA (available as Versene 1:5000 from GIBCO-BRL). Cells were washed in 10 milliliters phosphate buffered saline (available from GIBCO-BRL) and plated in 96 well black clear bottom plates (available from Costar, Cambridge, Mass.). The cells were incubated at 37° C. in 5% carbon dioxide for about 16 hours. The cells were then treated with the FLIPR® Calcium Assay Reagent contained in the FLIPR® Calcium No Wash Assay Dye Kit as per Molecular Devices' directions for measuring calcium in CHO-K1 cells. The cells were used to screen for muscarinic receptor modulators by adding a final concentration of 30 uM of experimental compound to the cells loaded with the FLIPR® Calcium Assay Reagent using the FLIPR® as per Molecular Devices' instructions. The fluorescence of the cells was measured by the FLIPR® 384 Fluorometric Imaging Plate Reader system over a period of two minutes. Agonists were identified by the determination of percent of stimulation of fluorescence induced by the experimental compound as compared to the fluorescent signal generated by the muscarinic agonist, carbamoylcholine chloride at a concentration of 1 mM. Background fluorescence generated by the solvating agent, dimethyl sulfoxide at a final concentration of 0.6% volume/volume, was subtracted from the carbamoylcholine chloride and experimental compound treated cell signals. For determining modulators which reduce the activity of the receptor such as antagonists, experimental compounds were added prior to the treatment of the cells with a concentration of carbamoylcholine chloride inducing about 50% of the signal at 1 mM carbamoylchloline chloride. In this case, percent inhibition was calculated as the fluorescence signal generated by carbamoylcholine chloride in the presence of experimental compound as a percentage of the fluorescence signal generated by cells treated with carbamoylcholine chloride in the absence of experimental compound.

1. A substantially pure protein having the amino acid sequence selected from the group consisting of: SEQ ID NO: 2, a mutant thereof, a fragment thereof, SEQ ID NO: 4, a mutant thereof, and a fragment thereof.

2. The protein of claim 1 wherein said protein has the amino acid sequence selected from the group consisting of: SEQ ID NO: 2, a mutant thereof, and a fragment thereof.

3. An isolated nucleic acid molecule that comprises a nucleic acid sequence that encodes the protein of claim 1.

4. A recombinant expression vector comprising the nucleic acid molecule of claim 3.

5. A host cell comprising the recombinant expression vector of claim 4.

6. An isolated nucleic acid molecule having a nucleic acid sequence selected from the group consisting of: SEQ ID NO: 1, a fragment thereof having at least 10 nucleotides, SEQ ID NO: 3, and a fragment thereof having at least 10 nucleotides.

7. An isolated nucleic acid molecule consisting of SEQ ID NO: 1 or a fragment thereof having at least 10 nucleotides.

8. A recombinant expression vector comprising the nucleic acid molecule of claim 6.

9. A host cell comprising the recombinant expression vector of claim 8.

10. An isolated antibody which binds to an epitope on SEQ ID NO: 2 or an epitope on SEQ ID NO: 4.

11. A method of identifying a modulator of a hemipteran muscarinic receptor protein activity comprising the steps of:
performing a test assay by contacting a test cell, which comprises a recombinant expression vector that contains a nucleic acid sequence that encodes said hemipteran muscarinic receptor, wherein said test cell expresses hemipteran muscarinic receptor, with a solution containing calcium in the presence of a test compound, and detecting the amount of intracellular calcium in said test cell;
performing a negative control assay by contacting a negative control cell which comprises a recombinant expression vector that contains a nucleic acid sequence that encodes said hemipteran muscarinic receptor, wherein said negative control cell expresses hemipteran muscarinic receptor, with a solution containing calcium in the absence of said test compound, and detecting the amount of intracellular calcium in said negative control cell;
comparing the amount of intracellular calcium in said test cell to the amount of intracellular calcium in said negative control cell
wherein a change in the amount of and a hemipteran muscarinic receptor agonist calcium in said test cell compared to the amount of intracellular calcium in said negative control cell indicates the test compound is a modulator of a hemipteran muscarinic receptor protein activity.

12. The method of claim 11 wherein the intracellular calcium is detected by using an assay in which fluorescence generated by dye inside of said cell interacting with intracellular calcium is measured.

13. The method of claim 11 further comprising performing a positive control assay by contacting a positive control cell which comprises a recombinant expression vector that contains a nucleic acid sequence that encodes said hemipteran muscarinic receptor, wherein said positive control cell expresses hemipteran muscarinic receptor, with a solution containing calcium in the absence of said test compound and in the presence of a hemipteran muscarinic receptor agonist, and detecting the amount of intracellular calcium in said positive control cell.

14. The method of claim 11 further comprising the step of
performing a second-type negative control assay by contacting a hemipteran muscarinic receptor negative control cell that does not express hemipteran muscarinic receptor with a solution containing calcium in the absence of said test compound, and detecting the amount of calcium taken up by said hemipteran muscarinic receptor negative control cell; and/or
performing a third-type negative control assay by contacting a hemipteran muscarinic receptor negative control cell that does not express hemipteran muscarinic receptor with a solution containing calcium in the absence of said test compound and in the presence of a hemipteran muscarinic receptor agonist, and detecting the amount of calcium taken up by said hemipteran muscarinic receptor negative control cell.

15. The method of claim 11 wherein said hemipteran muscarinic receptor protein has an amino acid sequence selected from the group consisting of: SEQ ID NO: 2, a mutant thereof, a fragment thereof, SEQ ID NO: 4, a mutant thereof, and a fragment thereof.

16. The method of claim 15 wherein said hemipteran muscarinic receptor protein has an amino acid sequence selected from the group consisting of: SEQ ID NO: 2, a mutant thereof, and a fragment thereof.

17. A method of identifying an inhibitor of a hemipteran muscarinic receptor protein activity comprising the steps of:
performing a test assay by contacting a test cell, which comprises a recombinant expression vector that contains a nucleic acid sequence that encodes said hemipteran muscarinic receptor, wherein said test cell expresses hemipteran muscarinic receptor, with a solution containing calcium and a hemipteran muscarinic receptor agonist in the presence of a test compound, and detecting the amount of intracellular calcium in said test cell;
performing a control assay by contacting a negative control cell, which comprises a recombinant expression vector that contains a nucleic acid sequence that encodes said hemipteran muscarinic receptor, wherein said test cell expresses hemipteran muscarinic receptor, with a solution containing calcium and a hemipteran muscarinic receptor agonist in the absence of said test compound, and detecting the amount of intracellular calcium in said negative control cell;
comparing the amount of intracellular calcium in said test cell to the amount of intracellular calcium in said control cell
wherein a decrease in the amount of intracellular calcium in said test cell compared to the amount of intracellular calcium in said control cell indicates the test compound is an inhibitor of a hemipteran muscarinic receptor protein activity.

18. The method of claim 17 wherein the intracellular calcium is detected by using an assay in which fluorescence generated by dye inside of said cell interacting with intracellular calcium is measured.

19. The method of claim 17 wherein said hemipteran muscarinic receptor protein has an amino acid sequence selected from the group consisting of: SEQ ID NO: 2, a mutant thereof, a fragment thereof, SEQ ID NO: 4, a mutant thereof, and a fragment thereof.

20. A method of preparing an isolated protein having the amino acid sequence selected from the group consisting of: SEQ ID NO: 2, a mutant thereof, a fragment thereof, SEQ ID NO: 4, a mutant thereof, and a fragment thereof comprising the step of isolating said protein from a host cell of claim 8.


SEQUENCE LISTING (29 sequences)

SEQ ID NO: 1 - DNA, 1693 bp, Aphis gossypii
ccgcggttcacgctgtcgcagacggtgttcatcgcgctcgtgtccggcatgctcagcttcgtcaccgtgtccggcaacataatggtcatggtgtcgttcaagatcgacaagcagctgcagaacatcagcaactacttcctgttcagcctggccatggccgacttcttcatcgggctcatatccatgccgctgttcaccgtgtacaccatactggggtactggccgttcggccggcacgtgtgcgacggctggctggcgctcgactacttggccagcaacgcttccgtgctcaacttgctgctgatcagcttcgacaggtacctgtccgtcaccaggccgctgacgtacagggccaagcggaccaaaggcaaagcgttgatgttcatatcggctgcctggatcatttcgctcggcttatggccaccatggatttatctgtggccgatcatagaaggtcgtcggaccgtgcccgaaattcagtgttatatacagttcatactgaccaatcagtacattacatttggtacggccattgctgctttttacgtaccggtaacagtgatgtgttttctgtatttccgtatatacagagagacaaaaaagagacaaaaagacttgccgaatctgcaagccatgaataaagcacacaacgatcatcaacacgaggaaacgtggtgtcgcatacggtccgagtcgaatcaagcgaattcattggatcggcgggaattgtatgaaaccagttcattgcgcaaagcgtactctcagtgttcattgaaagcgaggcgcctgctgacgtggtcgtggctgcgcgattggtgcgtggactggtggcactcaggccgcgacgacgattacgatgaggaagacgaccagacgcccagcgacgtgccggggggcataagctacggtactaccgtgtccaggtcgacgtcgttgaacgtgatacatcaacagcagcccacaacgccgacgccgataaaatcgtatcacgcggtcaacaataacaaccgactgacccgccgatctttgtccagcgattcggtgtacacgatcgtgataaagttgccgggcgacagcgacccgcaagacgcgccgacagtgagacagtatcacgggtgttacggggaaacgcgcgcggcaaactgcaggatgacgccgtcgcagccggacgtccggctgccgctgatgaagatcgtgcatcagaagcagctgaacaaccccagggcgcagggagccgcgctccaggcgaaggaaaaaaagaagaaaaaattccaggagaagaagagcgaacggaaggcggccaaaacgctgtcggccatactgctgacgttcatcatcacgtggacgccgtataacatactggtgctgctgaaaccgttcacggcgcccacggggggtgacgacggggccggcggcggccacgaggccgacagcaacaaggagtgggtgacgaagggcatgtgggactttttctattacctatgctacatcaacagtaccatcaacccggtgtgctacgccctgtgcaacgccactttccgcatgacctacgtccggatactcaagtgcaagtggaacactcgcaggcagccgccgcacttccgtgcgtgatgaccgacctgcaccgaccggccgcggttaccgcgcatatatgtttatataatatattacttatatatatatatatatataa

SEQ ID NO: 2 - protein, 976 aa, APHIS GOSSYPII
PRFTLSQTVFIALVSGMLSFVTVSGNIMVMVSFKIDKQLQNISNYFLFSLAMADFFIGLISMPLFTVYTILGYWPFGRHVCDGWLALDYLASNASVLNLLLISFDRYLSVTRPLTYRAKRTKGKALMFISAAWIISLGLWPPWIYLWPIIEGRRTVPEIQCYIQFILTNQYITFGTAIAAFYVPVTVMCFLYFRIYRETKKRQKDLPNLQAMNKAHNDHQHEETWCRIRSESNQANSLDRRELYETSSLRKAYSQCSLKARRLLTWSWLRDWCVDWWHSGRDDDYDEEDDQTPSDVPGGISYGTTVSRSTSLNVIHQQQPTTPTPIKSYHAVNNNNRLTRRSLSSDSVYTIVIKLPGDSDPQDAPTVRQYHGCYGETRAANCRMTPSQPDVRLPLMKIVHQKQLNNPRAQGAALQAKEKKKKKFQEKKSERKAAKTLSAILLTFIITWTPYNILVLLKPFTAPTGGDDGAGGGHEADSNKEWVTKGMWDFFYYLCYINSTINPVCYALCNATFRMTYVRILKCKWNTRRQPPHFRALNLLLISFDRYLSVTRPLTYRAKRTKGKALMFISAAWIISLGLWPPWIYLWPIIEGRRTVPEIQCYIQFILTNQYITFGTAIAAFYVPVTVMCFLYFRIYRETKKRQKDLPNLQAMNKAHNDHQHEETWCRIRSESNQANSLDRRELYETSSLRKAYSQCSLKARRLLTWSWLRDWCVDWWHSGRDDDYDEEDDQTPSDVPGGISYGTTVSRSTSLNVIHQQQPTTPTPIKSYHAVNNNNRLTRRSLSSDSVYTIVIKLPGDSDPQDAPTVRQYHGCYGETRAANCRMTPSQPDVRLPLMKIVHQKQLNNPRAQGAALQAKEKKKKKFQEKKSERKAAKTLSAILLTFIITWTPYNILVLLKPFTAPTGGDDGAGGGHEADSNKEWVTKGMWDFFYYLCYINSTINPVCYALCNATFRMTYVRILKCKWNTRRQPPHFRA

SEQ ID NO: 3 - DNA, 1741 bp, Aphis gossypii
atgtacggaaaccagacgaacggcaccatcggcttcgagacgaagggaccgcggttcacgctgtcgcagacggtgttcatcgcgctcgtgtccggcatgctcagcttcgtcaccgtgtccggcaacataatggtcatggtgtcgttcaagatcgacaagcagctgcagaacatcagcaactacttcctgttcagcctggccatggccgacttcttcatcgggctcatatccatgccgctgttcaccgtgtacaccatactggggtactggccgttcggccggcacgtgtgcgacggctggctggcgctcgactacttggccagcaacgcttccgtgctcaacttgctgctgatcagcttcgacaggtacctgtccgtcaccaggccgctgacgtacagggccaagcggaccaaaggcaaagcgttgatgttcatatcggctgcctggatcatttcgctcggcttatggccaccatggatttatctgtggccgatcatagaaggtcgtcggaccgtgcccgaaattcagtgttatatacagttcatactgaccaatcagtacattacatttggtacggccattgctgctttttacgtaccggtaacagtgatgtgttttctgtatttccgtatatacagagagacaaaaaagagacaaaaagacttgccgaatctgcaagccatgaataaagcacacaacgatcatcaacacgaggaaacgtggtgtcgcatacggtccgagtcgaatcaagcgaattcattggatcggcgggaattgtatgaaaccagttcattgcgcaaagcgtactctcagtgttcattgaaagcgaggcgcctgctgacgtggtcgtggctgcgcgattggtgcgtggactggtggcactcaggccgcgacgacgattacgatgaggaagacgaccagacgcccagcgacgtgccggggggcataagctacggtactaccgtgtccaggtcgacgtcgttgaacgtgatacatcaacagcagcccacaacgccgacgccgataaaatcgtatcacgcggtcaacaataacaaccgactgacccgccgatctttgtccagcgattcggtgtacacgatcgtgataaagttgccgggcgacagcgacccgcaagacgcgccgacagtgagacagtatcacgggtgttacggggaaacgcgcgcggcaaactgcaggatgacgccgtcgcagccggacgtccggctgccgctgatgaagatcgtgcatcagaagcagctgaacaaccccagggcgcagggagccgcgctccaggcgaaggaaaaaaagaagaaaaaattccaggagaagaagagcgaacggaaggcggccaaaacgctgtcggccatactgctgacgttcatcatcacgtggacgccgtataacatactggtgctgctgaaaccgttcacggcgcccacggggggtgacgacggggccggcggcggccacgaggccgacagcaacaaggagtgggtgacgaagggcatgtgggactttttctattacctatgctacatcaacagtaccatcaacccggtgtgctacgccctgtgcaacgccactttccgcatgacctacgtccggatactcaagtgcaagtggaacactcgcaggcagccgccgcacttccgtgcgtgatgaccgacctgcaccgaccggccgcggttaccgcgcatatatgtttatataatatattacttatatatatatatatatataa

SEQ ID NO: 4 - protein, 112 aa, APHIS GOSSYPII
MYGNQTNGTIGFETKGPRFTLSQTVFIALVSGMLSFVTVSGNIMVMVSFKIDKQLQNISNYFLFSLAMADFFIGLISMPLFTVYTILGYWPFGRHVCDGWLALDYLASNASV

SEQ ID NO: 5 - DNA, 29 bp, Artificial Sequence
  Degenerate Primer
ggcaacgtgatggtgatgathwsnttyaa

SEQ ID NO: 6 - DNA, 28 bp, Artificial Sequence
  Degenerate Primer
ggtgggtctcgatgaactgdatrtarca

SEQ ID NO: 7 - DNA, 26 bp, Artificial Sequence
  Degenerate Primer
ccggtttctcgccagatnckccarta

SEQ ID NO: 8 - DNA, 29 bp, Artificial Sequence
  Degenerate Primer
ggcaacgtgatggtgatgathwsnttyaa

SEQ ID NO: 9 - DNA, 28 bp, Artificial Sequence
  Degenerate Primer
tagaaagcagccatagcgtnccraangt

SEQ ID NO: 10 - DNA, 26 bp, Artificial Sequence
  Degenerate Primer
ccggcggacgagtgytayathcartt

SEQ ID NO: 11 - DNA, 30 bp, Artificial Sequence
  Degenerate Primer
tggggttgatggtggagttnayrtarcana

SEQ ID NO: 12 - DNA, 28 bp, Artificial Sequence
  Degenerate Primer
ctccggaaggttgcgttganarngcrta

SEQ ID NO: 13 - DNA, 18 bp, Asphi Gossypii
cgagcgaaatgatccagg

SEQ ID NO: 14 - DNA, 23 bp, Aphis Gossypii
aggtacctgtcgaagctgatcag

SEQ ID NO: 15 - DNA, 16 bp, Aphis Gossypii
agcacggaagcgttgc

SEQ ID NO: 16 - DNA, 19 bp, Aphis Gossypii
cgacagcaacaaggagtgg

SEQ ID NO: 17 - DNA, 23 bp, Aphis Gossypii
ccagcgagaacaagaagtagttg

SEQ ID NO: 18 - DNA, 20 bp, Aphis Gossypii
cagggtattcgccaggatac

SEQ ID NO: 19 - DNA, 19 bp, Aphis Gossypii
tcatcaccatgacattgcc

SEQ ID NO: 20 - DNA, 24 bp, Aphis Gossypii
gggagtgagtaccgcggtcccttc

SEQ ID NO: 21 - DNA, 35 bp, Aphis Gossypii
agcgagctcgaggatccctattccaccatgtacgg

SEQ ID NO: 22 - DNA, 21 bp, Aphis Gossypii
cagcccaggttcacgctgtcg

SEQ ID NO: 23 - DNA, 19 bp, Aphis Gossypii
gcgcaatgaactggtttca

SEQ ID NO: 24 - DNA, 21 bp, Aphis Gossypii
cgtgttgatgatcgttgtgtg

SEQ ID NO: 25 - DNA, 18 bp, Aphis Gossypii
ggtcggtgcaggtcggtc

SEQ ID NO: 26 - DNA, 21 bp, Aphis Gossypii
ctgtgatgttcatatcggctg

SEQ ID NO: 27 - DNA, 18 bp, Aphis Gossypii
cacgcacggaagtgcggc

SEQ ID NO: 28 - DNA, 23 bp, Aphis Gossypii
tacgtaccggtaacagtgatgtg

SEQ ID NO: 29 - DNA, 43 bp, Aphis Gossypii
gtcgaagcttgaggattccctgcggtcatcacgcacggaagtg